(19) United States
(12) Patent Application Publication
(10) Pub. No.: US 2014/0171475 A1
(43) Pub. Date: Jun. 19, 2014

(54) ANIMAL ECTOPARASITE-CONTROLLING AGENT

(71) Applicant: Kaori Ikari, Takarazuka-shi (JP)

(72) Inventor: Kaori Ikari, Takarazuka-shi (JP)

(73) Assignee: SUMITOMO CHEMICAL COMPANY, LIMITED, Tokyo (JP)

(21) Appl. No.: 14/123,028

(22) PCT Filed: May 15, 2012
(86) PCT No.: PCT/JP2012/062883

§ 371 Date: Nov. 27, 2013

(30) Foreign Application Priority Data
May 31, 2011 (JP) 2011-121558

(51) Int. Cl. A61K 31/42

(52) U.S. Cl. CPC A61K 31/42
USPC 514/378

(57) ABSTRACT

An animal ectoparasite-controlling agent comprising a benzylamide compound represented by the formula (1): wherein R³ represents a hydrogen atom; a halogen atom; a cyano group; a C1-C6 alkyl group; a C1-C6 haloalkyl group; a C1-C6 alkoxy group or a C1-C6 haloalkoxy group; and R⁴ represents a C1-C6 alkyl group substituted with one or more C1-C6 alkoxy group(s); a C1-C6 alkyl group substituted with one or more cyano group(s); a C1-C6 alkyl group; a C1-C6 haloalkyl group or a C3-C6 cycloalkyl group, as an active ingredient and an inert carrier, has an excellent controlling effect on animal ectoparasites.

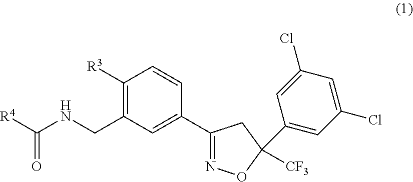

### TECHNICAL FIELD

The present invention relates to animal ectoparasite-controlling agents and methods for controlling animal ectoparasites.

### BACKGROUND ART

Heretofore, various compounds for controlling parasites living on the body surface or hair of animals or in the vicinity thereof have been found, and methods for controlling the parasites comprising applying agents containing said compounds to the body surface of animals or orally administrating the agents to animals have been developed (see, for example, Patent literature 1). However, conventional compounds are not always sufficiently effective, and thus there is still a demand for agents comprising compounds having excellent controlling effects on animal ectoparasites.

### PRIOR ART REFERENCE

### Patent Literature

Patent literature 1: JP-A-2003-313104

### SUMMARY OF INVENTION

### Technical Problem

The object of the present invention is to provide an animal ectoparasite-controlling agent having excellent controlling effects on animalectoparasites.

### Solution to Problem

The inventor of the present invention has intensively studied for attaining the above object, and finally found that an agent containing a benzylamide compound represented by the following formula (1) as an active ingredient shows excellent controlling effects on animal ectoparasites, thereby reaching the present invention.

Namely, the present invention includes the followings:

[1] An animal ectoparasite-controlling agent comprising a benzylamide compound represented by the formula (1):

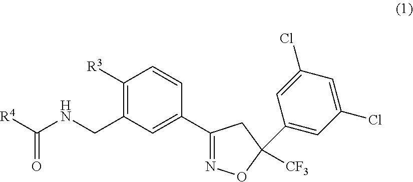

wherein R³ represents a hydrogen atom; a halogen atom; a cyano group; a C1-C6 alkyl group; a C1-C6 haloalkyl group; a C1-C6 alkoxy group or a C1-C6 haloalkoxy group; and R⁴ represents a C1-C6 alkyl group substituted with one or more C1-C6 alkoxy group(s); a C1-C6 alkyl group substituted with one or more cyano group(s); a C1-C6 alkyl group; a C1-C6 haloalkyl group or a C3-C6 cycloalkyl group (hereinafter referred as “the present benzylamide compound”),

as an active ingredient and an inert carrier (hereinafter referred to as “the controlling agent of the present invention”).

[2] The animal ectoparasite-controlling agent according to the item [1], which is in the form of an oral formulation or an external formulation for skin.

[3] The animal ectoparasite-controlling agent according to the item [1], which is in the form of a liquid formulation.

[4] The animal ectoparasite-controlling agent according to the item [1], which is in the form of capsule formulation, a tablet or a chewable tablet.

[5] The animal ectoparasite-controlling agent according to the item [1], wherein the animal ectoparasite is a flea or a tick.

[6] A method for controlling an animal ectoparasite, which comprises applying an effective amount of the present benzylamide compound to an animal.

[7] The method for controlling an animal ectoparasite according to the item [6], wherein the present benzylamide compound is orally administered.

[8] The method for controlling an animal ectoparasite according to the item [6], wherein the present benzylamide compound is externally applied to a skin.

[9] The method for controlling an animal ectoparasite according to the item [8], wherein the present benzylamide compound is applied by spot-on application or pour-on application.

[10] The method for controlling an animal ectoparasite according to any one of the items [6] to [9], wherein the animal is a dog or a cat.

[11] The method for controlling an animal ectoparasite according to any one of the items [6] to [9], wherein the animal is a cow, a horse, a pig or a sheep.

[12] The method for controlling an animal ectoparasite according to any one of the items [6] to [11], wherein the animal ectoparasite is a flea or a tick.

### Advantageous Effects of Invention

The controlling agent of the present invention has excellent controlling effects on animal ectoparasites.

### DESCRIPTION OF EMBODIMENTS

In this description, “a halogen atom” represents a fluorine atom, a chlorine atom, a bromine atom or an iodine atom.

Also, in this description, “C3-C6” described in “a C3-C6 cycloalkyl group” represents that the overall number of carbon atoms forming the cycloalkyl group is within a range of 3 to 6.

Examples of the “C1-C6 alkyl group” used herein include a methyl group, an ethyl group, a propyl group, an isopropyl group, a butyl group, an isobutyl group, a sec-butyl group, a tert-butyl group, a pentyl group, a 1-methylbutyl group, a 2-methylbutyl group, a 3-methylbutyl group, a 1-ethylpropyl group, a 1,1-dimethylpropyl group, a 1,2-dimethylpropyl group, a 2,2-dimethylpropyl group, a hexyl group, a 1-methylpentyl group, a 2-methylpentyl group, a 1,1-dimethylbutyl group and a 1,3-dimethylbutyl group.

Examples of the “C1-C6 haloalkyl group” used herein include a fluoromethyl group, a difluoromethyl group, a dichloromethyl group, a trifluoromethyl group, a chlorofluoromethyl group, a bromofluoromethyl group, a chlorodifluoromethyl group, a bromodifluoromethyl group, a 1-fluoroethyl group, a 1,1-difluoroethyl group, a 2,2,2-trifluoroethyl group, a 1,1,2,2,2-pentafluoroethyl group, a 3,3,3-trifluoropropyl group, a 1,1,2,2,3,3,3-heptafluoropropyl group, a 4,4,4-trifluorobutyl group and a 1,2,2,2-tetrafluoro-1-(trifluoromethyl)ethyl group.

Examples of the “C3-C6 cycloalkyl group” used herein include a cyclopropyl group, a 1-methylcyclopropyl group, a 2-methylcyclopropyl group, 2,2-dimethylcyclopropyl group, a cyclobutyl group, cyclopentyl group, a 1-methylcyclopentyl group, a 2-methylcyclopentyl group, a 3-methylcyclopentyl group and a cyclohexyl group.

Examples of the “C1-C6 alkoxy group” used herein include a, methoxy group, an ethoxy group, a propoxy group, an isopropoxy group, a butoxy group, an isobutoxy group, a sec-butoxy group, a tert-butoxy group, a pentyloxy group, a 1-methylbutoxy group, a 2-methylbutoxy group, a 3-methylbutoxy group, a 1-ethylpropoxy group, a 1,1-dimethylpropoxy group, a 1,2-dimethylpropoxy group, a 2,2-dimethylpropoxy group, a hexyloxy group, a 1-methylpentyloxy group, a 2-methylpentyloxy group, a 1,1-dimethylbutoxy group and a 1,3-dimethylbutoxy group.

Examples of the “C1-C6 haloalkoxy group” used herein include a difluoromethoxy group, a trifluoromethoxy group, a chlorodifluoromethoxy group, a bromodifluoromethoxy group, a 2-fluoroethoxy group, a 2-chioroethoxy group, a 2,2,2-trifluoroethoxy group, a 1,1,2,2-tetrafluoroethoxy group, a 2-chloro-1,1,2-trifluoroethoxy group, a 2-bromo-1,1,2-trifluoroethoxy group, a 1,1,2,2,2-pentafluoroethoxy group, a 2,2-dichloro-1,1,2-trifluoroethoxy group, a 2,2,2-trichloro-1,1-difluoroethoxy group, 2-bromo-1,1,2,2-tetrafluoroethoxy group, a 2,2,3,3-tetrafluoropropyloxy group, a 1,1,2,3,3,3-hexafluoropropyloxy group, a 2,2,2-trifluoro-1-(trifluoromethyl)ethoxy group, a 1,1,2,2,3,3,3-heptafluoropropyloxy group and a 2-bromo-1,1,2,3,3,3-hexafluoropropyloxy group.

Examples of the present benzylamide compound include the following benzylamide compounds:

a benzylamide compound represented by the formula (1), wherein R³ is a hydrogen atom;

a benzylamide compound represented by the formula (1), wherein R³ is a C1-C6 alkyl group;

a benzylamide compound represented by the formula (1), wherein R³ is a C1-C6 haloalkyl group;

a benzylamide compound represented by the formula (1), wherein R³ is a halogen atom;

a benzylamide compound represented by the formula (1), wherein R³ is a fluorine atom;

a benzylamide compound represented by the formula (1), wherein R³ is a chlorine atom;

a benzylamide compound represented by the formula (1), wherein R³ is a bromine atom;

a benzylamide compound represented by the formula (1), wherein R³ is an iodine atom;

a benzylamide compound represented by the formula (1), wherein R³ is a cyano group;

a benzylamide compound represented by the formula (1), wherein R³ is a C1-C6 alkoxy group;

a benzylamide compound represented by the formula (1), wherein R³ is a C1-C6 haloalkoxy group;

a benzylamide compound represented by the formula (1), wherein R⁴ is a C1-C6 alkyl group;

a benzylamide compound represented by the formula (1), wherein R⁴ is a C1-C6 haloalkyl group;

a benzylamide compound represented by the formula (1), wherein R⁴ is a C3-06 cycloalkyl group;

a benzylamide compound represented by the formula (1), wherein R⁴ is a C1-C6 alkyl group substituted with one or more C1-C6 alkyl group(s);

a benzylamide compound represented by the formula (1), wherein R⁴ is a C1-C6 alkyl group substituted with one or more cyano group(s);

a benzylamide compound represented by the formula (1), wherein R³ is a C1-C6 alkyl group and R⁴ is a C1-C6 alkyl group;

a benzylamide compound represented by the formula (1), wherein R³ is a C1-C6 alkyl group and R⁴ is a C1-C6 haloalkyl group;

a benzylamide compound represented by the formula (1), wherein R³ is a C1-C6 alkyl group and R⁴ is a C3-06 cycloalkyl group;

a benzylamide compound represented by the formula (1), wherein R³ is a C1-C6 alkyl group and R⁴ is a C1-C6 alkyl group substituted with one or more C1-C6 alkoxy group(s);

a benzylamide compound represented by the formula (1), wherein R³ is a C1-C6 alkyl group and R⁴ is a C1-C6 alkyl group substituted with one or more cyano group(s);

a benzylamide compound represented by the formula (1), wherein R³ is a C1-C6 haloalkyl group and R⁴ is a C1-C6 alkyl group;

a benzylamide compound represented by the formula (1), wherein R³ is a C1-C6 haloalkyl group and R⁴ is a C1-C6 haloalkyl group;

a benzylamide compound represented by the formula (1), wherein R³ is a C1-C6 haloalkyl group and R⁴ is a C3-06 cycloalkyl group;

a benzylamide compound represented by the formula (1), wherein R³ is a C1-C6 haloalkyl group and R⁴ is a C1-C6 alkyl group substituted with one or more C1-C6 alkyl group(s);

a benzylamide compound represented by the formula (1), wherein R³ is a C1-C6 haloalkyl group and R⁴ is a C1-C6 alkyl group substituted with one or more cyano group(s);

a benzylamide compound represented by the formula (1), wherein R³ is a C1-C6 alkoxy group and R⁴ is a C1-C6 alkyl group;

a benzylamide compound represented by the formula (1), wherein R³ is a C1-C6 alkoxy group and R⁴ is a C1-C6 haloalkyl group;

a benzylamide compound represented by the formula (1), wherein R³ is a C1-C6 alkoxy group and R⁴ is a C3-06 cycloalkyl group;

a benzylamide compound represented by the formula (1), wherein R³ is a C1-C6 alkoxy group and R⁴ is a C1-C6 alkyl group substituted with one or more C1-C6 alkoxy group(s);

a benzylamide compound represented by the formula (1), wherein R³ is a C1-C6 alkoxy group and R⁴ is a C1-C6 alkyl group substituted with one or more cyano group(s);

a benzylamide compound represented by the formula (1), wherein R³ is a C1-C6 haloalkoxy group and R⁴ is a C1-C6 alkyl group;

a benzylamide compound represented by the formula (1), wherein R³ is a C1-C6 haloalkoxy group and R⁴ is a C1-C6 haloalkyl group;

a benzylamide compound represented by the formula (1), wherein R³ is a C1-C6 haloalkoxy group and R⁴ is a C3-06 cycloalkyl group;

a benzylamide compound represented by the formula (1), wherein R³ is a C1-C6 haloalkoxy group and R⁴ is a C1-C6 alkyl group substituted with one or more C1-C6 alkoxy group(s);

a benzylamide compound represented by the formula (1), wherein R³ is a C1-C6 haloalkoxy group and R⁴ is a C1-C6 alkyl group substituted with one or more cyano group(s);

a benzylamide compound represented by the formula (1), wherein R³ is a halogen atom and R⁴ is a C1-C6 alkyl group, a C1-C6 haloalkyl group, a C3-C6 cycloalkyl group, a C1-C6 alkyl group substituted with one or more C1-C6 alkoxy group(s) or a C1-C6 alkyl group substituted with one or more cyano group(s);

a benzylamide compound represented by the formula (1), wherein R³ is a halogen atom and R⁴ is a C1-C6 alkyl group;

a benzylamide compound represented by the formula (1), wherein R³ is a halogen atom and R⁴ is a C1-C6 haloalkyl group;

a benzylamide compound represented by the formula (1), wherein R³ is a halogen atom and R⁴ is a C3-C6 cycloalkyl group;

a benzylamide compound represented by the formula (1), wherein R³ is a halogen atom and R⁴ is a C1-C6 alkyl group substituted with one or more C1-C6 alkoxy group(s);

a benzylamide compound represented by the formula (1), wherein R³ is a halogen atom and R⁴ is a C1-C6 alkyl group substituted with one or more cyano group(s);

a benzylamide compound represented by the formula (1), wherein R³ is a fluorine group and R⁴ is a C1-C6 alkyl group;

a benzylamide compound represented by the formula (1), wherein R³ is a fluorine group and R⁴ is a C1-C6 haloalkyl group;

a benzylamide compound represented by the formula (1), wherein R³ is a fluorine group and R⁴ is a C3-06 cycloalkyl group;

a benzylamide compound represented by the formula (1), wherein R³ is a fluorine group and R⁴ is a C1-C6 alkyl group substituted with one or more C1-C6 alkyl group(s);

a benzylamide compound represented by the formula (1), wherein R³ is a fluorine group and R⁴ is a C1-C6 alkyl group substituted with one or more cyano group(s);

a benzylamide compound represented by the formula (1), wherein R³ is a chlorine atom and R⁴ is, a C1-C6 alkyl group, a C1-C6 haloalkyl group, a C3-06 cycloalkyl group, a C1-C6 alkyl group substituted with one or more C1-C6 alkyl group(s) and a C1-C6 alkyl group substituted with one or more cyano group(s);

a benzylamide compound represented by the formula (1), wherein R³ is a chlorine atom and R⁴ is a C1-C6 alkyl group;

a benzylamide compound represented by the formula (1), wherein R³ is a chlorine atom and R⁴ is a C1-C6 haloalkyl group;

a benzylamide compound represented by the formula (1), wherein R³ is a chlorine atom and R⁴ is a C3-06 cycloalkyl group;

a benzylamide compound represented by the formula (1), wherein R³ is a chlorine atom and R⁴ is a C1-C6 alkyl group substituted with one or more C1-C6 alkoxy group(s); and

a benzylamide compound represented by the formula (1), wherein R³ is a chlorine atom and R⁴ is a C1-C6 alkyl group substituted with one or more cyano group(s).

Hereinafter, methods for producing the present benzylamide compound will be explained:

The present benzylamide compound can be produced, for example, by the following Production methods 1 and 2.

### Production Method 1:

The present benzylamide compound can be produced by reacting the compound (2) with the compound (3):

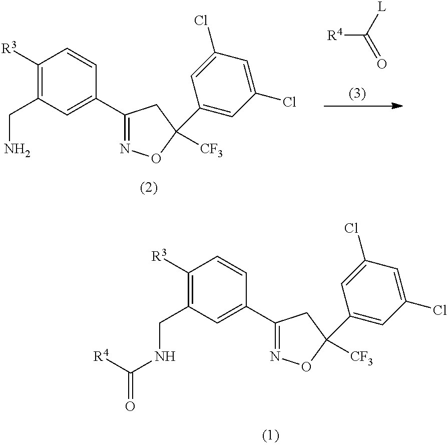

wherein, R³ and R⁴ are as defined above and L represents a hydroxyl group or a chlorine atom.

The reaction is generally performed in a solvent.

Examples of the solvent to be used in the reaction include ethers such as tetrahydrofuran, diethyl ether, tert-butyl methyl ether, ethylene glycol dimethyl ether and 1,4-dioxane; acid amides such as N,N-dimethylformamide; nitriles such as acetonitrile; aromatic hydrocarbons such as toluene and xylene; esters such as ethyl acetate; sulfoxides such as dimethyl sulfoxide; sulfolane; halogenated hydrocarbons such as 1,2-dichloroethane, chloroform and chlorobenzene; and mixture thereof.

When L is a chlorine atom, the reaction is generally performed in the presence of a base.

Examples of the base to be used in the reaction include alkali metal hydrides such as sodium hydride; carbonates such as potassium carbonate; alkali metal alkoxides such as potassium tert-butoxide; and organic amines such as triethylamine and pyridine.

When L is a hydroxyl group, the reaction is performed in the presence of a condensation agent.

Examples of the condensation agent to be used in the reaction include dicyclohexylcarbodiimide and 1-(3-dimethylaminopropyl)-3-ethyl carbodiimide hydrochloride.

In the reaction, the amount of the compound (3) to be used is generally 1 to 10 mol relative to 1 mol of the compound (2), and the amount of the base or the condensation agent to be used is generally 1 to 10 mol relative to 1 mol of the compound (2).

The reaction temperature is generally within a range of 0 to 100° C., and the reaction time is generally within a range of 0.5 to 24 hours.

After the reaction is completed, the reaction mixture may be worked up, for example, by extraction with an organic solvent, drying and concentration, to isolate the present benzylamide compound. The isolated present benzylamide compound may be further purified, for example, by chromatography, recrystallization or the like.

### Production Method 2:

The present benzylamide compound can be produced by reacting the compound (2) with the compound (21):

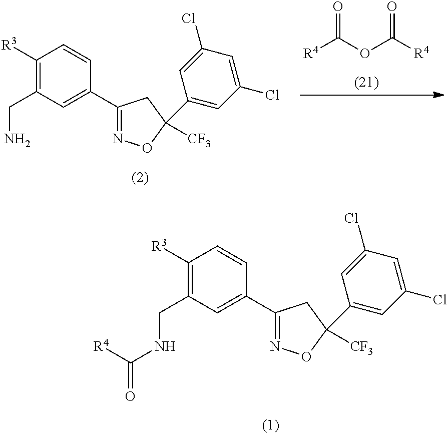

wherein, R³ and R⁴ are as defined above.

The reaction is optionally performed in a solvent.

Examples of the solvent to be used in the reaction include ethers such as tetrahydrofuran, diethyl ether, tert-butyl methyl ether, ethylene glycol dimethyl ether and 1,4-dioxane; acid amides such as N,N-dimethylformamide; nitriles such as acetonitrile; aromatic hydrocarbons such as toluene and xylene; esters such as ethyl acetate; sulfoxides such as dimethyl sulfoxide; sulfolane; halogenated hydrocarbons such as 1,2-dichloroethane, chloroform and chlorobenzene; and mixture thereof.

The reaction is optionally performed in the presence of a base.

Examples of the base to be used in the reaction include alkali metal hydrides such as sodium hydride; carbonates such as potassium carbonate; alkali metal alkoxides such as potassium tert-butoxide; and organic bases such as triethylamine, pyridine, 4-(dimethyl amino)pyridine and imidazole.

In the above reaction, the amount of the compound (21) to be used is generally 1 to 10 mol relative to 0.1 mol of the compound (2), and if appropriate, may be used as a solvent. If the above base is needed, the amount of the base to be used is generally 1 to 10 mol relative to 1 mol of the compound (2).

The reaction temperature is generally within a range of 0 to 100° C., and the reaction time is generally within a range of 0.5 to 24 hours.

After the reaction is completed, the reaction mixture may be worked up, for example, by extraction with an organic solvent, drying and concentration, to isolate the present benzylamide compound. The isolated present benzylamide compound may be further purified, for example, by chromatography, recrystallization or the like.

The compound (2) can be produced, for example, by a method described in International patent publication No. WO2010/032437.

Examples of the animal ectoparasites to be controlled by the controlling agent of the present invention include as follows:

Fleas (Aphaniptera): Pulex spp. such as human flea (Pulex irritans); Ctenocephalides spp. such as cat flea (Ctenocephalides felis) and dog flea (Ctenocephalides canis); Xenopsylla spp. such as oriental rat flea (Xenopsylla cheopis); Tunga spp. such as chigoe (Tunga penetrans); Echidnophaga spp. such as chicken flea (Echidnophaga gallinacea); Nosopsyllus spp. such as European mouse flea (Nosopsyllus fasciatus); and the like.

Lice (Anoplura): Pediculus spp. such as head louse (Pediculus humanus capitis); Phtirus spp. such as crab louse (Pthirus pubis); Haematopinus spp. such as cattle louse (Haematopinus eurysternus) and hog louse (Haematopinus suis); Damalinia spp. such as sheep louse (Dalmalinia ovis) and Damalinia bovis; Linognathus spp. such as long nosed cattle louse (Linognathus vituli) and sheep face louse (Linognathus ovillus); Solenopotes spp. such as little blue cattle louse (Solenopotes capillatus); and the like.

Mallophages: Menopon spp. such as chicken louse (Menopon gallinae); Trimenopon spp.; Trinoton spp.; Trichodectes spp. such as dog biting louse (Trichodectes canis); Felicola spp. such as cat louse (Felicola subrostratus); Bovicola spp. such as cattle biting louse (Bovicola bovis); Menacanthus spp. such as chicken body louse (Menacanthus stramineus); Werneckiella spp.; and Lepikentron spp.; and the like.

Hemiptera: Cimix spp. such as bedbug (Cimex lectularius) and tropical bedbug (Cimex hemipterus); Reduvius spp. such as Reduvius senilis; Arilus spp. such as Arilus critatus; Rhodnius spp. such as Rhodnius prolixus; Triatoma spp. such as triatomine bug (Triatoma rubrofasciata); Panstrongylus ssp.; and the like.

Ticks (Acarina): Amblyomma spp. such as lone star tick (Amblyomma americanum) and Ambryomma maculatum; Boophilus spp. such as cattle tick (Boophilus microplus) and Boophilus annulatus; Dermacentor spp. such as American dog tick (Dermacentor variabilis), Dermacentor taiwanicus and Dermacentor andersoni; Haemaphysalis spp. such as bush tick (Haemaphysalis longicornis), Haemaphysalis flava and Haemaphysalis campanulata; Ixodes spp. such as Ixodes ovatus, taiga tick (Ixodes persulcatus), black legged tick (Ixodes scapularis), western black-legged tick (Ixodes pacificus) and Ixodes holocyclus; Rhipicephalus spp. such as brown dog tick (Rhipicephalus sanguineus) and Rhipicephalus appendiculatus; Argas spp. such as fowl tick (Argas persicus); Ornithodorus spp. such as Ornithodorus hermsi and Ornithodorus turicata; psoroptid mites such as sheep scab mite (Psoroptes ovis) and horse psoroptic mange mite (Psoroptes equi); Knemidocoptes spp. such as Knemidocoptes mutans; Notoedres spp. such as cat mange mite (Notoedres cati) and rat ear mange mite (Notoedres muris); Sarcoptes spp. such as itch mite (Sarcoptes scabiei); Otodectes spp. such as ear mite (Octodectes cynotis); Listrophorus such as rabbit fur mite (Listrophorus gibbus); Chorioptes spp.; Hypodectes spp.; Pterolichus spp.; Cytodites spp.; Laminosioptes spp.; Dermanyssus spp. such as parasitoid mite (Dermanyssus gallinae); Ornithonyssus spp. such as northern fowl mite (Ornithonyssus sylviarum) and house tick (Ornithonyssus bacoti); Varroa spp. such as honey bee mite (Varroa jacobsoni); Cheyletiella spp. such as dog cheyletid mite (Cheyletiella yasguri) and cat mite (Cheyletiella blakei); Ornithocheyletia spp.; Demodex spp. such as dog follicle mite (Demodex canis) and cat follicle mite (Demodex cati); Myobia spp.; Psorergates spp.; Trombicula spp. such as trombiculid mite (Trombicula akamushi), Trombicula pallida and Trombicula scutellaris.

Among these animal ectoparasites, fleas, lice and ticks are particularly exemplified.

The target animals to which the controlling agent of the present invention is applied are generally those to be hosts for the above animal ectoparasites, and include, for example, homeothermic animals and heterothermic animals that are reared as livestock or pets. Examples of the homeothermic animals include mammals such as cow, water buffalo, sheep, goat, pig, camel, deer, fallow deer, reindeer, horse, donkey, dog, cat, rabbit, ferret, mouse, rat, hamster, squirrel, and monkey; fur-bearing animals such as mink, chinchilla, and raccoon; and birds such as chicken, goose, turkey, duck, pigeon, parrot, and quail. Examples of the heterothermic animals include reptiles such as land turtle, sea turtle, Trachemys scripta, Reeve's pond turtle, lizard, iguana, chameleon, gecko, python, Colubridae, and cobra; and fish such as freshwater fish and salt-water fish, e.g., trout, carp, and eel. Preferred are homeothermic animals, and more preferred are mammals such as dog, cat, cow, horse, pig, sheep, and goat.

In the controlling agent of the present invention, the present benzylamide compound may be used alone, but is generally formulated with inert carriers such as solid carriers and liquid carriers, and optionally other formulation additives such as surfactants and the like. The controlling agent of the present invention is usually a formulation obtained by mixing the present benzylamide compound with inert carriers such as solid carriers and liquid carriers, and optionally adding thereto surfactants or other formulation additives. Examples of the formulation include liquid formulations such as emulsifiable concentrate, oil formulation, oily liquid formulation, aqueous liquid formulation, solution, shampoo, and suspension formulation; dusts; granules; paste formulation; cream; ointment; microencapsulated formulation; foaming formulation; aerosol formulation; carbon dioxide gas formulation; tablets; chewable tablets; bolus formulation; capsule formulation; animal feed premixe; syrup; sheet formulation, film-type formulation; resin formulation; injection formulation; implanted formulation; and suppository formulation. The suitable formulation is chosen when the present invention is employed.

The controlling agent of the present invention generally contains the present benzylamide compound in an amount of 0.001 to 99.9% by weight of the whole composition.

Examples of the solid carriers to be used in the formulation include natural or synthetic minerals such as clay, kaolin, talc, bentonite, sericite, quartz, sulfur, activated carbon, calcium carbonate, diatomaceous earth, pumice, calcite, sepiolite, white mica, silica, alumina, vermiculite, and perlite; small granules such as sawdust, corn spike, coconut shell, and tobacco stem; gelatin; vaseline; methylcellose; lanolin; lard; cocoa butter; and the like.

Examples of the liquid carriers include alcohols such as methanol, ethanol, isopropyl alcohol, butanol, and hexanol; polyhydric alcohols such as ethylene glycol, propylene glycol, dipropylene glycol, 1,3-butylene glycol, glycerine, and polyethylene glycol; ethers such as diethyl ether, ethyleneglycol dimethyl ether, ethyleneglycol monomethyl ether, diethyleneglycol monomethyl ether, diethyleneglycol monoethyl ether, propyleneglycol monomethyl ether, dipropyleneglycol monomethyl ether, tetrahydrofuran, and dioxane; esters such as ethyl acetate, butyl acetate, and propylene carbonate; fatty acid esters such as diisopropyl adipate, diisobutyl adipate, and isopropyl myristate; ketones such as acetone, methyl ethyl ketone, methyl isobutyl ketone, and cyclohexanone; aromatic or aliphatic hydrocarbons such as xylene, toluene, alkylnaphthalene, phenylxylylethane, kerosene, gas oil, hexane, cyclohexane, and liquid paraffin; sulfoxides such as dimethyl sulfoxide; acid amides such as N,N-dimethylformamide and N,N-dimethylacetoamide; N-methyl-2-pyrrolidone, γ-butyrolactone; vegetable oils such as soybean oil, cottonseed oil, castor oil, and palm oil; plant essential oil such as orange oil, hyssop oil, and lemon oil; silicone oils such as dimethyl silicone oil, high-molecular-weight dimethyl silicone oil, cyclic silicone oil, polyether modified silicone oil, amino modified silicone oil, and methylphenyl silicone oil; water and the like.

Examples of the surfactants include nonionic surfactants, amphotetic surfactants, anionic surfactants, and cationic surfactants, specifically as follows:

Nonionic surfactants: sorbitan fatty acid esters such as sorbitan stearate, and sorbitan oleate; glycerine fatty acid esters such as glyceryl stearate, glyceryl isostearate, glyceryl oleate, polyglyceryl stearate, polyglyceryl isostearate, and polyglyceryl oleate; polyoxyethylene alkyl ethers such as polyoxyethylene lauryl ether, polyoxyethylene cetyl ether, polyoxyethylene stearyl ether, polyoxyethylene oleyl ether, and polyoxyethylene styryl phenyl ether; polyoxyethylene sorbitan fatty acid esters such as polyoxyethylene sorbitan coconut oil fatty acid esters, polyoxyethylene sorbitan oleate, and polyoxyethylene sorbitan stearate; polyoxyethylene sorbit fatty acid esters such as polyoxyethylene sorbit tetraoleate; polyoxyethylene-cured castor oil, alkylphenol polyglycol ether; and the like.

Ampholytic surfactants: betaines such as laurylbetaine and stearylbetaine; imidazoline derivatives such as disodium, N-lauryl-p-iminodipropionate; lecithins; and the like.

Anionic surfactants: alkyl sulfates such as sodium lauryl sulfate and triethanolamine lauryl sulfate; polyoxyethylene alkyl ether sulfates such as sodium lauryl polyoxyethylene ether sulfate and triethanolamine polyoxyethylene lauryl ether sulfate; alkylbenzene sulfonate such as sodium dodecylbenzene sulfonate;

polyoxyethylene alkyl ether phosphates such as sodium dipolyoxyethylene lauryl ether phosphate and sodium dipolyoxyethylene oleyl ether phosphate.

Cationic surfactants: alkyl ammonium salts such as cetyltrimethyl ammonium chloride, and distearyl dimethyl ammonium chloride.

Other formulation additives include, for example, dispersing agents, antioxidants, coloring agents, light stabilizers, adhesives, and the like.

Examples of the dispersing agents include lignin sulfonate and methylcellulose.

Examples of the antioxidants include BHT and BHA.

Examples of the coloring agents include food tar colors such as Red No. 2 (Amaranth), Red No. 3 (Erythrosine), Yellow No. 4 (Tartrazine), Green No. 3 (Fast Green FCF), and Blue No. 1 (Brilliant Blue FCF); iron oxide, titanium oxide, Prussian blue, alizarin dyes, azo dyes, and phthalocyanine dyes.

Examples of the light stabilizers include benzophenone compounds, benzoate compounds, and benzotriazole compounds.

Examples of the adhesives include bentonite, colloidal silicic acid, cellulose derivatives, starch derivatives, polyacrylates, natural polymers, alginic acid salts, and gelatin.

Examples of binders in the tablets and chewable tablets include methylcellulose, carboxymethylcellulose, ethylhydroxyethylcellulose; protein derivatives such as zein and gelatin; synthetic polymers such as polyvinyl alcohol and polyvinyl pyrrolidone; starch, and celluloses.

In addition, the tablets and chewable tablets may contain fillers such as starch, microcrystalline cellulose, sugar, and lactose; lubricants such as magnesium stearate and talc; disintegrants such as starch, cellulose, and carbonates.

The tablets can be produced by, for example, mixing the present benzylamide compound, binders and the like, and compressing the resulting mixture to a suitable size. Tablets may be coated, if desired. Examples of the coating agent to be used for coating tablets include those containing acetic acid-phthalic acid cellulose, diethyl phthalate, ethanol, and dichloromethane; those containing hydroxypropylcellulose, polyethylene glycol, water, and titanium dioxide; enteric film coating agents such as polyvinyl acetal diethylaminoacetate; and other film forming materials such as food coloring agents, and hydroxypropyl methylcellulose containing aqueous or non-aqueous solvents. The film coating agents may contain plasticizers or coloring agents.

Examples of propellants for the foaming formulation, aerosol formulation, or carbon dioxide gas formulation include propane gases, butane gas, Freon gases, liquefied petroleum gases, dimethyl ether, and carbon dioxide gases.

Examples of bases for the resin formulation include vinyl chloride polymers, ethylene copolymers, polyurethanes, polyethylenes, polypropylenes, and polyethylene terephthalate. The bases optionally contain phthalic acid esters such as dimethyl phthalate and dioctyl phthalate; and plasticizers such as adipic acid esters and stearic acid. The resin formulation can be produced by kneading the present benzylamide compound into the base with a common kneading machine, and then forming the mixture by injection molding, extrusion molding, press molding, or the like. If necessary, the molded products may be further processed by shape forming, cutting, or the like to obtain animal ear tags and the like.

Examples of capsules for the capsule formulation include gelatin capsules and hydroxypropyl methylcellulose capsules.

Examples of, bases for the suppository include cacao butter, laurin butter, polyethylene glycol, glycerogelatin, sodium stearate, witepsol, and their mixtures.

The controlling agent of the present invention may be used in mixture or combination with commonly known other insecticides, agents for killing animal parasitic mites, or agents for killing endoparasites. In addition, the controlling agent of the present invention may also be used in mixture or combination with repellents.

The animal ectoparasite-controlling method according to the present, invention (hereinafter referred to as the controlling method of the present invention) comprises applying an effective amount of the present benzylamide compound to an animal.

According to the controlling method of the present invention, the present benzylamide compound can therapeutically, suppressively, prophylactically or protectively act on animal ectoparasites.

By the controlling method of the present invention, animal ectoparasites may be suppressed systemically or nonsystemically. The controlling method of the present invention can be applied to animal ectoparasites at all or any developmental stages.

In the controlling method of the present invention, the present benzylamide compound can be administered orally or parenterally to a host animal. Examples of the oral administration method include the administration of the present benzylamide compound in the form of an oral formulation to an animal. Examples of the parenteral administration method include the application of the present benzylamide compound in the form of an external preparation for skin, injection formulation, suppository, implanted formulation, or resin formulation in suitable shape such as collar or ear tag to an animal.

### (1) Oral Administration:

In the controlling method of the present invention, the present benzylamide compound may be orally administered to an animal in the form of, for example, liquid formulations such as emulsifiable concentrate, oil solutions, oily liquid formulation, aqueous liquid formulation, solution, suspension formulation; gel; dusts; granules; paste formulation; tablets; chewable tablets; bolus formulation; capsule formulation; animal feed premix; or syrup.

### (2) Parenteral Administration:

### (a) External Application to Skin:

In the controlling method of the present invention, the present benzylamide compound may be externally applied to the skin of an animal, for example, in the form of liquid formulations such as emulsifiable concentrate, oil solution, oily liquid formulation, aqueous liquid formulation, solution, shampoo, or suspension formulation; dusts; cream; ointment; aerosol formulation, or sheet formulation, by spot-on application, pour-on application, immersing, spraying, coating, bathing, washing, rubbing, dispersing, or the like. Preferred application methods are spot-on application and pour-on application.

The spot-on application generally means the dropping or coating application of a liquid formulation onto the skin from head to tail of a host animal.

The pour-on application generally means the pouring application of a liquid formulation along the back line of a host animal.

In this case, the present benzylamide compound can be formulated into a liquid formulation by using the above liquid carriers.

### (b) Injection Application:

In the controlling method of the present invention, the present benzylamide compound in the form of injection formulation may be applied to an animal by intraruminal injection, intramuscular injection, intravenous injection, or subcutaneous injection.

### (c) Other Applications:

In the controlling method of the present invention, the present benzylamide compound may be applied to an animal in the form of a suppository, implanted formulation, or resin formulation in suitable shape such as collar or ear tag.

The amount of the present benzylamide compound to be applied to an animal may vary depending on the type of the target animal or animal ectoparasite to be controlled, but is generally 1 to 5000 mg/kg-living body weight of the animal. When the present benzylamide compound is orally administered or applied by injection, the amount is preferably 1 to 100 mg/kg. When the present benzylamide compound is externally applied to skin, the amount is preferably 1 to 100 mg/kg. In the oral administration, the amount is more preferably 1 to 50 mg/kg, and most preferably 5 to 50 mg/kg. In the external application to the skin, the amount is more preferably 1 mg/kg to 100 mg/kg, and most preferably 5 to 50 mg/kg.

Examples of the present benzylamide compound specifically include as follows;

A compound represented by the formula (1a):

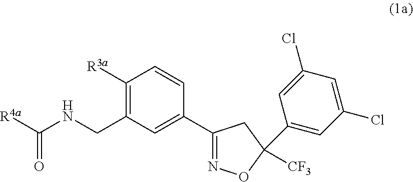

wherein, a combination of R^{3a} and R^{4a} represents any one of the combinations described in Table 1.

TABLE 1

| The present | | |
|---|---|---|
| benzylamide | | |
| compound | R^{3a} | R^{4a} |
| (1) | hydrogen | ethyl |
| (2) | hydrogen | methyl |
| (3) | fluorine | propyl |
| (4) | fluorine | 1-methylethyl |
| (5) | fluorine | cyclopropyl |
| (6) | fluorine | 1,1-dimethylethyl |
| (7) | fluorine | cyclopropylmethyl |
| (8) | fluorine | trifluoromethyl |
| (9) | fluorine | 2,2,2-trifluoroethyl |
| (10) | fluorine | 2-methoxypropyl |
| (11) | fluorine | 2-cyanoethyl |
| (12) | chlorine | ethyl |
| (13) | chlorine | cyclopropyl |
| (14) | chlorine | 2-methoxypropyl |
| (15) | chlorine | 2-cyanoethyl |
| (16) | bromine | ethyl |
| (17) | bromine | cyclopropyl |
| (18) | bromine | 2-methoxypropyl |
| (19) | bromine | 2-cyanoethyl |
| (20) | iodine | ethyl |
| (21) | methyl | ethyl |
| (22) | cyano | ethyl |
| (23) | trifluoromethyl | ethyl |
| (24) | trifluoromethyl | 2-methoxypropyl |
| (25) | trifluoromethyl | 2-methoxypropyl |
| (26) | methoxy | ethyl |
| (27) | hydrogen | 2,2,2-trifluoroethyl |
| (28) | chlorine | methyl |
| (29) | chlorine | 3,3,3-trifluoropropyl |
| (30) | chlorine | methoxymethyl |
| (31) | chlorine | 1-methylethyl |
| (32) | chlorine | propyl |
| (33) | chlorine | 2-methylpropyl |
| (34) | chlorine | cyanomethyl |
| (35) | chlorine | 3-bromopropyl |
| (36) | chlorine | trifluoromethyl |
| (37) | chlorine | cyclobutyl |
| (38) | chlorine | cyclopentyl |
| (39) | fluorine | methyl |

### EXAMPLES

Hereinafter, the present invention will be explained in detail with reference to Reference Production Examples of the present benzylamide compound, Formulation Examples of the controlling agents of the present invention and Test Examples, but the present invention should not be interpreted to be limited to these examples.

First, Production Examples of the present benzylamide compound was described as follows.

### Reference Production Example 1

In 2 mL of tetrahydrofuran, 200 mg of 2-chloro-5-[5-(3,5-dichlorophenyl)-5-trifluoromethyl-4,5-dihydroisoxazol-3-yl]benzylamine and 58 mg of triethylamine were dissolved, and 60 mg of cyclopropanecarbonyl chloride was dropwised therein under ice-cooling, then the mixture was stirred for 1.5 hours at room temperature. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 222 mg of N-{2-chloro-[5-(3,5-dichlorophenyl)-5-trifluoromethyl-4,5-dihydroisoxazol-3-yl]benzyl}cyclopropanecarboxylic amide (hereinafter, referred to as the present benzylamide compound (13)).

The present benzylamide compound (13)

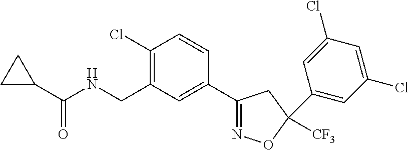 [SMILES: O=C(NCc1cc(C2=NOC(c3cc(Cl)cc(Cl)c3)(C(F)(F)F)C2)ccc1Cl)C1CC1]

¹H-NMR (CDCl₃) δ: 7.62 (1H, d), 7.58 (1H, dd), 7.49 (2H, d), 7.43-7.41 (2H, m), 6.17 (1H, br s), 4.55 (2H, d), 4.06 (1H, d), 3.68 (1H, d), 1.43-1.36 (1H, m), 1.00-0.98 (2H, m), 0.79-0.77 (2H, m).

### Reference Production Example 2

In 2 mL of tetrahydrofuran, 200 mg of 2-chloro-5-[5-(3,5-dichlorophenyl)-5-trifluoromethyl-4,5-dihydroisoxazol-3-yl]benzylamine and 58 mg of triethylamine were dissolved, and 45 mg of acetyl chloride was dropwised therein under ice-cooling, then the mixture was stirred for 0.5 hour at room temperature. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 207 mg of N-{2-chloro-[5-(3,5-dichlorophenyl)-5-trifluoromethyl-4,5-dihydroisoxazol-3-yl]benzyl}acetamide (hereinafter, referred to as the present benzylamide compound (28)).

The present benzylamide compound (28)

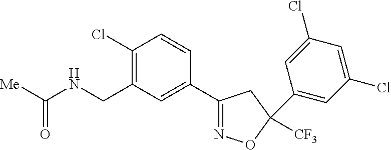 [SMILES: CC(=O)NCc1cc(C2=NOC(c3cc(Cl)cc(Cl)c3)(C(F)(F)F)C2)ccc1Cl]

¹H-NMR (CDCl₃) δ: 7.64 (1H, d), 7.59 (1H, dd), 7.50 (2H, d), 7.44-7.42 (2H, m), 6.02-5.99 (1H, br m), 4.52 (2H, d), 4.07 (1H, d), 3.68 (1H, d), 2.04 (3H, s)

### Reference Production Example 3

In 2 mL of tetrahydrofuran, 200 mg of 2-chloro-5-[5-(3,5-dichlorophenyl)-5-trifluoromethyl-4,5-dihydroisoxazol-3-yl]benzylamine and 58 mg of triethylamine were dissolved, and 91 mg of 4,4,4-trifluorobutyryl chloride was dropwised therein under ice-cooling, then the mixture was stirred for 4 hours at room temperature. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 207 mg of N-{2-chloro-[5-(3,5-dichlorophenyl)-5-trifluoromethyl-4,5-dihydroisoxazol-3-yl]benzyl}-4,4,4-trifluorobutanamide (hereinafter, referred to as the present benzylamide compound (29)).

The present benzylamide compound (29)

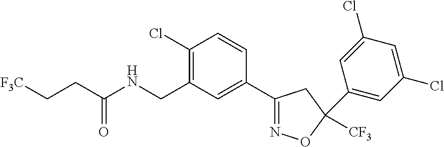 [SMILES: O=C(CCC(F)(F)F)NCc1cc(C2=NOC(c3cc(Cl)cc(Cl)c3)(C(F)(F)F)C2)ccc1Cl]

¹H-NMR (CDCl₃) δ: 7.61-7.59 (2H, m), 7.49 (2H, d), 7.45-7.42 (2H, m), 6.05 (1H, br s), 4.55 (2H, d), 4.05 (1H, d), 3.67 (1H, d), 2.58-2.45 (4H, m).

### Reference Production Example 4

In 2 mL of tetrahydrofuran, 212 mg of 2-chloro-5-[5-(3,5-dichlorophenyl)-5-trifluoromethyl-4,5-dihydroisoxazol-3-yl]benzylamine and 61 mg of triethylamine were dissolved, and 65 mg of methoxyasetyl chloride was dropwised therein under ice-cooling, then the mixture was stirred for 4 hours at room temperature. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 223 mg of N-{2-chloro-[5-(3,5-dichlorophenyl)-5-trifluoromethyl-4,5-dihydroisoxazol-3-yl]benzyl}methoxyacetamide (hereinafter, referred to as the present benzylamide compound (30)).

The present benzylamide compound (30)

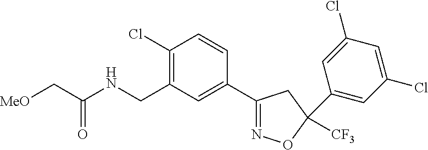 [SMILES: COCC(=O)NCc1cc(C2=NOC(c3cc(Cl)cc(Cl)c3)(C(F)(F)F)C2)ccc1Cl]

¹H-NMR (CDCl₃) δ: 7.65 (1H, d), 7.59 (1H, dd), 7.50 (2H, d), 7.44-7.43 (2H, m), 7.06-7.04 (1H, br m), 4.59 (2H, d), 4.07 (1H, d), 3.95 (2H, s), 3.69 (1H, d), 3.43 (3H, s).

### Reference Production Example 5

In 3 mL of tetrahydrofuran, 250 mg of 2-chloro-5-[5-(3,5-dichlorophenyl)-5-trifluoromethyl-4,5-dihydroisoxazol-3-yl]benzylamine and 105 mg of triethylamine were dissolved, and 111 mg of isobutyryl chloride was dropwised therein under ice-cooling, then the mixture was stirred for 0.5 hour under ice-cooling. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 151 mg of N-{2-chloro-[5-(3,5-dichlorophenyl)-5-trifluoromethyl-4,5-dihydroisoxazol-3-yl]benzyl}isobutyramide (hereinafter, referred to as the present benzylamide compound (31)).

The present benzylamide compound (31)

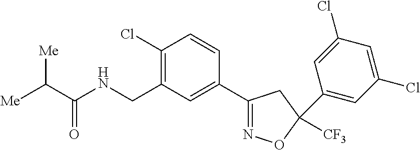 [SMILES: CC(C)C(=O)NCc1cc(C2=NOC(c3cc(Cl)cc(Cl)c3)(C(F)(F)F)C2)ccc1Cl]

¹H-NMR (CDCl₃) δ: 7.62 (1H, d), 7.59 (1H, dd), 7.50 (2H, d), 7.44-7.42 (2H, m), 5.96 (1H, br m), 4.52 (2H, d), 4.06 (1H, d), 3.68 (1H, d), 2.44-2.37 (1H, m), 1.19 (3H, d), 1.17 (3H, d).

### Reference Production Example 6

In 3 mL of tetrahydrofuran, 250 mg of 2-chloro-5-[5-(3,5-dichlorophenyl)-5-trifluoromethyl-4,5-dihydroisoxazol-3-yl]benzylamine and 105 mg of triethylamine were dissolved, and 111 mg of propionyl chloride was dropwised therein under ice-cooling, then the mixture was stirred for 0.5 hour under ice-cooling. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 249 mg of N-{2-chloro-[5-(3,5-dichlorophenyl)-5-trifluoromethyl-4,5-dihydroisoxazol-3-yl]benzyl}propionamide (hereinafter, referred to as the present benzylamide compound (32)).

The present benzylamide compound (32)

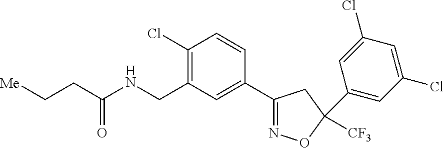 [SMILES: CCCC(=O)NCc1cc(C2=NOC(c3cc(Cl)cc(Cl)c3)(C(F)(F)F)C2)ccc1Cl]

¹H-NMR (CDCl₃) δ: 7.62-7.59 (2H, m), 7.51-7.48 (2H, m), 7.44-7.42 (2H, m), 5.96-5.93 (1H, br m), 4.53 (2H, d), 4.06 (1H, d), 3.68 (1H, d), 2.21 (2H, t), 1.71-1.64 (2H, m), 0.95 (3H, t).

### Reference Production Example 7

In 3 mL of tetrahydrofuran, 250 mg of 2-chloro-5-[5-(3,5-dichlorophenyl)-5-trifluoromethyl-4,5-dihydroisoxazol-3-yl]benzylamine and 105 mg of triethylamine were dissolved, and 126 mg of isovaleryl chloride was dropwised therein under ice-cooling, then the mixture was stirred for 0.5 hour under ice-cooling. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 235 mg of N-{2-chloro-[5-(3,5-dichlorophenyl)-5-trifluoromethyl-4,5-dihydroisoxazol-3-yl]benzyl}isovaleramide (hereinafter, referred to as the present benzylamide compound (33)).

The present benzylamide compound (33)

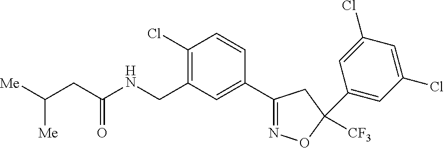 [SMILES: CC(C)CC(=O)NCc1cc(C2=NOC(c3cc(Cl)cc(Cl)c3)(C(F)(F)F)C2)ccc1Cl]

¹H-NMR (CDCl₃) δ: 7.61-7.60 (2H, m), 7.50-7.48 (2H, m), 7.43-7.41 (2H, m), 5.95-5.92 (1H, br m), 4.53 (2H, d z), 4.05 (1H, dd), 3.68 (1H, d), 2.13-2.06 (3H, m), 0.95 (6H, d,).

### Reference Production Example 8

In 3 mL of tetrahydrofuran, 250 mg of 2-chloro-5-[5-(3,5-dichlorophenyl)-5-trifluoromethyl-4,5-dihydroisoxazol-3-yl]benzylamine and 105 mg of triethylamine were dissolved. Then, 89 mg of cyanoacetic acid and 199 mg of 1-(3-dimethylaminopropyl)-3-ethylcarbodiimide hydrochloride were dropwised therein at room temperature, and the mixture was stirred for 2 hours at room temperature. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 235 mg of N-{2-chloro-[5-(3,5-dichlorophenyl)-5-trifluoromethyl-4,5-dihydroisoxazol-3-yl]benzyl}cyanoacetamide (hereinafter, referred to as the present benzylamide compound (34)).

The present benzylamide compound (34)

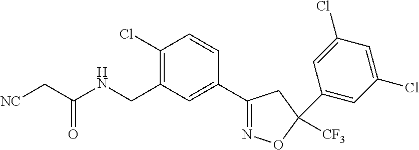 [SMILES: N#CCC(=O)NCc1cc(C2=NOC(c3cc(Cl)cc(Cl)c3)(C(F)(F)F)C2)ccc1Cl]

¹H-NMR (CDCl₃) δ: 7.65 (1H, d), 7.60 (1H, dd,), 7.49 (2H, s), 7.45 (1H, d), 7.42 (1H, t), 6.76-6.73 (1H, br m), 4.58 (2H, d), 4.07 (1H, d), 3.70 (1H, d), 3.43 (2H, s).

### Reference Production Example 9

In 2 mL of tetrahydrofuran, 250 mg of 2-chloro-5-[5-(3,5-dichlorophenyl)-5-trifluoromethyl-4,5-dihydroisoxazol-3-yl]benzylamine and 90 mg of triethylamine were dissolved, and 189 mg of trifluoroacetic anhydride was dropwised therein under ice-cooling, then the mixture was stirred for 3 hours at room temperature. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 280 mg of N-{2-chloro-[5-(3,5-dichlorophenyl)-5-trifluoromethyl-4,5-dihydroisoxazol-3-yl]benzyl}trifluoroacetamide (hereinafter, referred to as the present benzylamide compound (36)).

The present benzylamide compound (36)

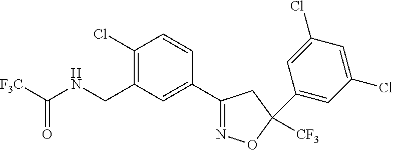 [SMILES: O=C(NCc1cc(C2=NOC(c3cc(Cl)cc(Cl)c3)(C(F)(F)F)C2)ccc1Cl)C(F)(F)F]

¹H-NMR (CDCl₃) δ: 7.66-7.64 (2H, m), 7.50-7.43 (4H, m), 6.80 (1H, br s), 4.64 (2H, d), 4.06 (1H, d), 3.68 (1H, d).

### Reference Production Example 10

In 2 mL of tetrahydrofuran, 254 mg of 2-chloro-5-[5-(3,5-dichlorophenyl)-5-trifluoromethyl-4,5-dihydroisoxazol-3-yl]benzylamine and 91 mg of triethylamine were dissolved, and 109 mg of cyclobutanecarbonyl chloride was dropwised therein under ice-cooling, then the mixture was stirred for 0.5 hour under ice-cooling. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 285 mg of N-{2-chloro-[5-(3,5-dichlorophenyl)-5-trifluoromethyl-4,5-dihydroisoxazol-3-yl]benzyl}cyclobutanecarboxamide (hereinafter, referred to as the present benzylamide compound (37)).

The present benzylamide compound (37)

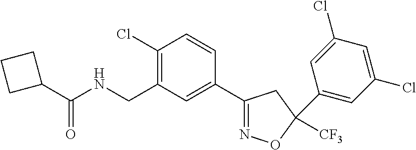 [SMILES: O=C(NCc1cc(C2=NOC(c3cc(Cl)cc(Cl)c3)(C(F)(F)F)C2)ccc1Cl)C1CCC1]

¹H-NMR (CDCl₃) δ: 7.62 (1H, s), 7.59 (1H, dd), 7.50 (2H, s), 7.43-7.41 (2H, m), 5.87-5.85 (1H, br m), 4.52 (2H, d), 4.06 (1H, d), 3.69 (1H, d), 3.08-3.00 (1H, m), 2.28-2.19 (4H, m), 1.96-1.91 (2H, m).

### Reference Production Example 11

In 2 mL of tetrahydrofuran, 254 mg of 2-chloro-5-[5-(3,5-dichlorophenyl)-5-trifluoromethyl-4,5-dihydroisoxazol-3-yl]benzylamine and 91 mg of triethylamine were dissolved, and 119 mg of cyclopentanecarbonyl chloride was dropwised therein under ice-cooling, then the mixture was stirred for 0.5 hour under ice-cooling. To the reaction mixture, an aqueous saturated sodium hydrogen carbonate solution was added and extracted with ethyl acetate. The organic layer was dried over anhydrous sodium sulfate and concentrated under reduced pressure. The resulting residue was subjected to silica gel column chromatography to obtain 261 mg of N-{2-chloro-[5-(3,5-dichlorophenyl)-5-trifluoromethyl-4,5-dihydroisoxazol-3-yl]benzyl}cyclopentanecarboxamide (hereinafter, referred to as the present benzylamide compound (38)).

The present benzylamide compound (38)

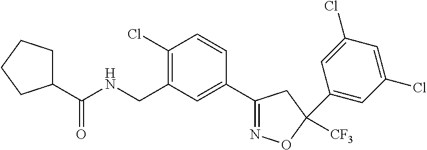 [SMILES: O=C(NCc1cc(C2=NOC(c3cc(Cl)cc(Cl)c3)(C(F)(F)F)C2)ccc1Cl)C1CCCC1]

¹H-NMR (CDCl₃) δ: 7.61 (1H, d), 7.58 (1H, dd), 7.49 (2H, d), 7.42-7.41 (2H, m), 6.02-6.00 (1H, br m), 4.52 (2H, d), 4.06 (1H, d), 3.68 (1H, d), 2.58-2.54 (1H, m), 1.86-1.64 (8H, m).

Hereinafter, Formulation Examples of the controlling agent of the present invention will be described:

### Formulation Example 1

### Tablets

Any one of the present benzylamide compounds (1) to (39) (100 mg), lactose (68.75 mg), a corn starch (237.5 mg), a microcrystalline cellulose (43.75 mg), a polyvinyl pyrrolidone (18.75 mg), a sodium carboxymethyl starch (28.75 mg), and magnesium stearate (2.5 mg) are mixed together, and the resulting mixture is compressed into tablets of suitable size.

### Formulation Example 2

### Tablets

Any one of the present benzylamide compound (1) to (39) (25 mg), D-mannitol (73 mg), a corn starch (30 mg), a low-substituted hydroxypropyl cellulose (7 mg), an aqueous 5% hydroxypropyl cellulose solution (appropriate amount), and magnesium stearate (appropriate amount) are mixed together, and the resulting mixture is compressed into tablets of suitable size.

### Formulation Example 3

### Tablets

Any one of the present benzylamide compounds (1) to (39) (400 mg), a corn starch (50 mg), a croscarmellose sodium (25 mg), lactose (120 mg), and magnesium stearate (5 mg) are mixed together, and the resulting mixture is compressed into tablets of suitable size.

### Formulation Example 4

### Tablets

Any one of the present benzylamide compounds (1) to (39) (60 mg), a microcrystalline cellulose (45 mg), a polyvinyl pyrrolidone (4 mg), a carboxymethyl starch sodium (4.5 mg), magnesium stearate (0.5 mg), and a talc (1 mg) are mixed together, and the resulting mixture is compressed into tablets of suitable size.

### Formulation Example 5

### Tablets

Any one of the present benzylamide compounds (1) to (39) (10 mg), a starch (15 mg), lactose (127 mg), a carboxymethylcellulose calcium (15 mg), magnesium stearate (1 mg), and a talc (2 mg) are mixed together, and the resulting mixture is compressed into tablets of suitable size.

### Formulation Example 6

### Tablets

Any one of the present benzylamide compounds (1) to (39) (100 mg), a dextrin (600 mg), a potato starch (200 mg), an animal feed powder (60 mg), a sesame oil (20 mg), and water (20 mg) are mixed together, and the resulting mixture is compressed into tablets of suitable size.

### Formulation Example 7

### Tablets

Any one of the present benzylamide compounds (1) to (39) (100 mg), lactose (33 mg), a corn starch (16 mg), a carboxymethylcellulose calcium (12 mg), a methylcellulose (6 mg), and magnesium stearate (2 mg) are mixed together, and the resulting mixture is compressed into tablets of suitable size.

### Formulation Example 8

### Tablets

Any one of the present benzylamide compounds (1) to (39) (10 mg), Fine Particles for Direct Compressing No. 209 (manufactured by Fuji Chemical Industry Co., Ltd.) (46.6 mg), magnesium aluminometasilicate (20%), a corn starch (30%), lactose (50%), a crystal cellulose (24 mg), a carboxymethylcellulose calcium (4 mg), and magnesium stearate (0.4 mg) are mixed together, and the resulting mixture is compressed into tablets of suitable size.

### Formulation Example 9

### Tablets

Any one of the present benzylamide compounds (1) to (39) (250 mg), magnesium stearate (4.5 mg), a corn starch (22.5 mg), a sodium starch glycolate (9 mg), lauryl sodium sulfate (4.5 mg), and a microcrystalline cellulose (159.5 mg) are mixed together, and the mixture is compressed into tablets of suitable size.

### Formulation Example 10

### Tablets

Any one of the present benzylamide compounds (1) to (39) (250 mg), lactose (101.5 mg), a wheat flour starch (6.5 mg), polyethylene glycol 6000 (5 mg), a talc (5 mg), magnesium stearate (2 mg), and deionized water (appropriate amount) are mixed together, and the mixture is compressed into tablets of suitable size.

### Formulation Example 11

### Tablets

Any one of the present benzylamide compounds (1) to (39) (200 mg), lactose (200 mg), a potato starch (266.5 mg), stearic acid (10 mg), a talc (217 mg), magnesium stearate (2.5 mg), a colloidal silica (32 mg), and ethanol (appropriate amount) are mixed together, and the mixture is compressed into tablets of suitable size.

### Formulation Example 12

### Tablets

Any one of the present benzylamide compounds (1) to (39) (50 mg), magnesium stearate (7.5 mg), and a microcrystalline cellulose (17.5 mg) are mixed together, and the mixture is compressed into tablets of suitable size.

### Formulation Example 13

### Tablets

Each of the tablets obtained by Formulation Examples 1 to 12 is coated with a coating agent containing a mixture of 20% acetic acid-phthalic acid cellulose, 3% diethyl phthalate, ethanol, and dichloromethane in equal volume to obtain the coated tablets.

### Formulation Example 14

### Tablets

Each of the tablets obtained by Formulation Examples 1 to 12 is coated with a coating agent obtained by dissolving hydroxypropyl cellulose 2910 (10.8 g) and polyethylene glycol 6000 (2.1 g) in a purified water (172.5 g) and dispersing thereinto titanium dioxide (2.1 g) to obtain the coated tablets.

### Formulation Example 15

### Capsule Formulation

Any one of the present benzylamide compounds (1) to (39) (25 mg), lactose (60 mg), a corn starch (25 mg), a carmellose calcium (6 mg), and 5% hydroxypropyl methylcellulose (appropriate amount) are mixed together, and the resulting mixture is filled into hard-shell gelatin capsules or hydroxypropyl methylcellulose capsules obtain a capsule formulation.

### Formulation Example 16

### Capsule Formulation

Any one of the present benzylamide compounds (1) to (39) (200 mg), lactose (148 mg), and magnesium stearate (2 mg) are mixed together, and the resulting mixture is filled into hard-shell gelatin capsules or hydroxypropyl methylcellulose capsules to obtain a capsule formulation.

### Formulation Example 17

### Capsule Formulation

Any one of the present benzylamide compounds (1) to (39) (250 mg), a dry starch (200 mg), and magnesium stearate (10 mg) are mixed together, and the resulting mixture is filled into hard-shell gelatin capsules or hydroxypropyl methylcellulose capsules to obtain a capsule formulation.

### Formulation Example 18

### Capsule Formulation

Any one of the present benzylamide compounds (1) to (39) (250 mg), a microcrystalline cellulose (400 mg), a fumed silicon dioxide (10 mg), and stearic acid (5 mg) are mixed together, and the resulting mixture is filled into hard-shell gelatin capsules or hydroxypropyl methylcellulose capsules to obtain a capsule formulation.

### Formulation Example 19

### Capsule Formulation

Any one of the present benzylamide compounds (1) to (39) (20 mg), lactose (251.8 mg), gelatin (2 mg), a corn starch (10 mg), talc (15 mg), and water (appropriate amount) are mixed together, and the resulting mixture is filled into hard-shell gelatin capsules or hydroxypropyl methylcellulose capsules to obtain a capsule formulation. Formulation Example 20: Oral suspension formulation

Any one of the present benzylamide compounds (1) to (39) (1000 mg), fumaric acid (500 mg), sodium chloride (2000 mg), methylparaben (150 mg), propylparaben (50 mg), a granulated sugar (25000 mg), sorbitol (70% solution; 13000 mg), VeegumK (Vanderbilt Co.; 100 mg), a fragrance (35 mg), a colorant (500 mg) and distillated water (added to the final volume of 100 mL) are mixed together to obtain an oral suspension formulation.

### Formulation Example 21

### Oral Suspension Formulation

Any one of the present benzylamide compounds (1) to (39) (50 mg), a carboxymethylcellulose sodium (50 mg), a syrup (1.25 ml), a benzoic acid solution (0.1 ml), a fragrance (appropriate amount), a colorant (appropriate amount) and distillated water (added to the final volume of 5 mL) are mixed together to obtain an oral suspension formulation.

### Formulation Example 22

### Oral Liquid Formulation

Any one of the present benzylamide compounds (1) to (39) (5% by weight) is dissolved in polysorbate 85 (5% by weight), benzyl alcohol (3% by weight), and propylene glycol (30% by weight). This solution is adjusted to pH 6.0 to 6.5 by adding a phosphate buffer, and thereto is added water to be a desired final volume to obtain an oral liquid formulation.

### Formulation Example 23

### Oral Liquid Formulation

Any one of the present benzylamide compounds (1) to (39) (10% by weight) is homogeneously dissolved in a corn oil (90% by weight) to obtain an oral liquid formulation.

### Formulation Example 24

### Oral Paste Formulation

Aluminum distearate (5% by weight) is dispersed with heating into a mixture of a distilled palm oil (57% by weight) and polysorbate 85 (3% by weight). This mixture is cooled to room temperature, and saccharine (25% by weight) is dispersed into the oil vehicle. To the mixture is added any one of the present benzylamide compounds (1) to (39) (10% by weight) to obtain an oral paste formulation.

### Formulation Example 25

### Granules for Oral Administration

Any one of the present benzylamide compounds (1) to (39) (5% by weight) is mixed with a lime stone powder (95% by weight), and the mixture is subjected to wet granulation to obtain granules for oral administration.

### Formulation Example 26

### Animal Feed Premix

Any one of the present benzylamide compounds (1) to (39) (0.15% by weight), an animal feed (95% by weight), and, a mixture (4.85% by weight) of dicalcium phosphate, a diatom earth, Aerosil, and a carbonate (or chalk) are sufficiently stirred and mixed to obtain an animal feed premix.

### Formulation Example 27

### Animal Feed Premix

Any one of the present benzylamide compounds (1) to (39) (0.15% by weight), Aerosil (2.5% by weight), a chalk (2.5% by weight), and an animal feed (94.85% by weight) are sufficiently stirred and mixed to obtain an animal feed premix.

### Formulation Example 28

### Liquid Formulation

Any one of the present benzylamide compounds (1) to (39) (20 g) is dissolved in diethylene glycol monoethyl ether (80 g) to obtain a liquid formulation.

### Formulation Example 29

### Liquid Formulation

Any one of the present benzylamide compounds (1) to (39) (20 g) is dissolved in propylene carbonate (80 g) to obtain a liquid formulation.

### Formulation Example 30

### Liquid Formulation

Any one of the present benzylamide compounds (1) to (39) (20 g) is dissolved in diisopropyl adipate (80 g) to obtain a liquid formulation.

### Formulation Example 31

### Liquid Formulation

Any one of the present benzylamide compounds (1) to (39) (20 g) is dissolved in diisobutyl adipate (80 g) to obtain a liquid formulation.

### Formulation Example 32

### Liquid Formulation

Any one of the present benzylamide compounds (1) to (39) (20 g) is dissolved in γ-butyrolactone (80 g) to obtain a liquid formulation.

### Formulation Example 33

### Liquid Formulation

Any one of the present benzylamide compounds (1) to (39) (20 g) is dissolved in a mixture of diethylene glycol monoethyl ether (40 g) and diisopropyl adipate (40 g) to obtain a liquid formulation.

### Formulation Example 34

### Liquid Formulation

Any one of the present benzylamide compounds (1) to (39) (20 g) is dissolved in a mixture of silicone oil (10 g) and diethylene glycol monoethyl ether (70 g) to obtain a liquid formulation.

### Formulation Example 35

### Emulsifiable Concentrate

Any one of the present benzylamide compounds (1) to (39) (5 g) is dissolved in a mixture of xylene (39.5 g) and N,N-dimethylformamide (39.5 g). To the mixture are added polyoxyethylene styryl phenyl ether (10 g) and calcium dodecylbenzenesulfonate (6 g), and the resulting mixture is stirred and mixed to obtain an emulsifiable concentrate.

### Formulation Example 36

### Shampoo

To any one of the present benzylamide compounds (1) to (39) (0.5 g) are added Nikkol TEALS-42 (manufactured by Nikko Chemicals Co., Ltd.; aqueous 42% triethanolamine lauryl sulfate solution; 60 g) and propylene glycol (20 g). The resulting mixture is sufficiently stirred and mixed to a homogeneous solution, and thereto is added water (19.5 g), and then the resulting mixture is sufficiently stirred and mixed to a shampoo as a homogeneous solution.

### Formulation Example 37

### Suppository

Any one of the present benzylamide compounds (1) to (39) (7.2 g) and Hosco S-55 (manufactured by Maruishi Pharmaceutical Co., Ltd.; 92.8 g) are dissolved and mixed at 100° C., and the resulting mixture is poured into a mold for suppository, and cooled and solidified to a suppository.

Hereinafter, Test Examples supporting an excellent controlling effect of the controlling agent of the present invention on animal ectoparasites will be described.

### Test Example 1

### Pesticidal Activity on Ticks (Haemaphysalis Longicornis) in Filter Paper Test

The present benzylamide compound (13) (5 mg) was dissolved in acetone (10 mL), and this acetone solution (1 mL) was uniformly applied onto one side of a filter paper (TOYO No. 2; 5×10 cm; the surface area of the filter paper was 50 cm², and thus the amount of the present benzylamide compound applied was 100 mg/m²). After drying, said filter paper was folded, and the both sides of the paper were clipped to form a bag. Into this bag, test ticks (Haemaphysalis longicornis, non-blood-fed young ticks, 10 ticks/group) were added, and the opening was clipped to seal the bag. Two (2) days later, the number of dead ticks was examined and the mortality was calculated by the following formula:

Mortality (%)=100×(number of dead ticks/number of ticks tested)

As a result, the present benzylamide compound (1) showed a mortality of 90% or more.

### Test Example 2

### Dropping Application Against Mouse-Infested Ticks (Haemaphysalis Longicornis)

The previous day of a dropping application, 30 test ticks (Haemaphysalis longicornis, young ticks) were deposited on a mouse. Before the dropping application, uninfested ticks were removed.

Any one of the present benzylamide compounds (12), (13), (15), (28), (29), (30), (31), (32), (33), (34), (36), (37) and (38) (5 mg) was dissolved in a mixture (5 mL) of propylene carbonate and diethylene glycol monoethyl ether in equal volume to prepare a 0.1% w/v solution. Said solution (200 μL) was applied dropwise to the whole body surface of a mouse with a pipette. To a control group, the mixture (200 μL) alone was applied. Said application was repeated 3 times per group.

Two (2) days after the application, the number of dead ticks was examined and the mortality was calculated by the following formula:

Mortality (%)=100×(number of dead ticks/infested ticks before dropping application)

As a result, each of the present benzylamide compounds (12), (13), (15), (28), (29), (30), (31), (32), (33), (34), (36), (37) and (38) showed a mortality of 60% or more.

### Test Example 3

### Oral Administration Against Mouse-Infested Ticks (Haemaphysalis Longicornis)

The previous day of an oral administration, 30 test ticks (Haemaphysalis longicornis, young ticks) were deposited on a mouse. Before the oral administration, uninfested ticks were removed.

The present benzylamide compound (13) (20 mg) was dissolved in dimethylformamide (680 mg), and thereto a corn oil was added to prepare a test solution (10 mL). Said test solution was orally administered to the mouse at the rate of 10 mL per 1 kg of the body weight of the mouse with a gastric sonde. To a control group, a 7% dimethylformamide/corn oil solution alone was orally administered. Each administration was repeated 3 times per group.

Two (2) days after the administration, the number of dead ticks was examined and the mortality was calculated by the following formula:

Mortality (%)=100×(number of dead ticks/infested ticks before oral administration)

As a result, the present benzylamide compound (13) showed a mortality of 70% or more.

### Test Example 4

### Oral Administration Against Mouse-Infested Fleas (Ctenocephalides felies)

The present benzylamide compound (13) (20 mg) was dissolved in dimethylformamide (680 mg), and thereto a corn oil was added to prepare a test solution (10 mL). Said test solution was orally administered to the mouse at the rate of 10 mL per 1 kg of the body weight of the mouse with a gastric sonde. To a control group, a 7% dimethylformamide/corn oil solution alone was orally administered. Each administration was repeated 3 times per group. After the administration, 20 test fleas (Ctenocephalides felies) were deposited on a mouse.

Two (2) days after the deposition, the number of dead fleas was examined and the mortality was calculated by the following formula:

Mortality (%)=100×(number of dead fleas/number of fleas tested)

As a result, the present benzylamide compound (13) showed a mortality of 70% or more.

### Test Example 5

### Dropping Application Against Dog-Infested Ticks (Haemaphysalis longicornis)

The previous day of a dropping application, 100 test ticks (Haemaphysalis longicornis, young ticks) were deposited on a dog (beagle). Before the dropping application, infested ticks were counted.

The present hydrazide compound (13) (1.5 g) was dissolved in diethylene glycol monoethyl ether (6.0 g) to prepare a test solution. Said test solution was directly dropped on the skin of the neck and back of the dog while pushing aside fur thereon at a rate of 0.1 ml per 1 kg of the dog's body weight (dose amount: 20 mg/kg). This is referred to as a test group. On the other hand, diethylene glycol monoethyl ether alone was applied dropwise to a placebo group.

Two (2) days after the application, the number of living ticks, which were infesting the dogs, was examined. When the examination was completed, all infested ticks were removed from the dogs.

Both of Forty (14) days and 28 days after the application, 100 test ticks (Haemaphysalis longicornis, young ticks) were repeatedly deposited on a dog (beagle) respectively. Two (2) days after the repeated deposition, the number of living ticks, which were infesting the dogs, was examined respectively. When each of the examination was completed, all infested ticks were removed from the dogs.

The infestation rate and control rate were calculated by the following formulae:

Method of calculating infestation rate and control rate at the initial stage (2 days) after the application:

Infestation rate (%) at X days after application=(number of living ticks at X days/number of living ticks before application)×100

Control rate (%) at X days after application {(infestation rate of test group before application infestation rate of test group at X days)/infestation rate of test group before application}×100

Method of calculating infestation rate and control rate at both 16 days and 30 days after the application:

Infestation rate (%) at X days after application=(number of living ticks at X days/number of living ticks repeatedly deposited)×100

Control rate (%) at X days after application {(infestation rate of placebo group at X days−infestation rate of test group at X days)/infestation rate of placebo group after X days}×100

In addition, if an infestation rate of a test group is higher than a placebo group, then the control rate is deemed to be 0%.

As a result, the present benzylamide compound (1) showed excellent tick control activities at a dose of 20 mg/kg at 16 days and 30 days after the application (Table 2).

TABLE 2

| | Tick control rates (%) at X days after the application | | |
|---|---|---|---|
| | X = 2 (days) | X = 16 (days) | X = 30 (days) |
| Compound (13) | 35 | 100 | 100 |

### Test Example 6

### Oral Administration Against Dog-Infested Ticks (Haemaphysalis longicornis)

The previous day of an oral administration, 100 test ticks (Haemaphysalis longicornis, young ticks) were deposited on a dog (beagle). Before the oral administration, infested ticks were counted.

A gelatin capsule in which the present benzylamide compound (13) was filled into a gelatin capsule so that the amount of the present benzylamide compound (1) administered was 40 mg per 1 kg of the dog's body weight was orally administered to the dog conpulsively. This is referred to as a test group. On the other hand, a geratin capsule alone was orally administered to a placebo group.

Two (2) days after the oral administration, the number of living ticks, which were infesting the dogs, was examined. When the examination was completed, all infested ticks were removed from the dogs.

The infestation rate and control rate were calculated by the following formulae:

Method of calculating infestation rate and control rate at the initial stage (2 days) after the application:

Infestation rate (%) at X days after application=(number of living ticks at X days/number of living ticks before application)×100

Control rate (%) at X days after application {(infestation rate of test group before application−infestation rate of test group at X days)/infestation rate of test group before application}×100

In addition, if an infestation rate of a test group is higher than a placebo group, then the control rate is deemed to be 0%.

As a result, the present benzylamide compound (13) showed excellent tick control activities at a dose of 40 mg/kg at 2 days after the administration (Table 3).

TABLE 3

| | Tick control rates (%) at 2 |
|---|---|
| | days after the administration |
| Compound (13) | 91 |

### Test Example 7

### Dropping Application Against Cat-Infested Fleas (Ctenocephalides felis)

The previous day of a dropping application, 50 test fleas (Ctenocephalides felis, adult ticks) were deposited on a cat. Before the dropping application, infested flaes were counted.

Each of the present benzylamide compound (13) (specified weight: 0.375 g, 0.75 g and 1.5 g) was dissolved in diethylene glycol monoethyl ether (6.0 g) to prepare a test solution. Said test solution was directly dropped on the skin of the neck and back of the cat while pushing aside fur thereon at a rate of 0.1 ml per 1 kg of the cat's body weight (dose amount: 5 mg/kg, 10 mg/kg and 20 mg/kg). This is referred to as a test group. On the other hand, diethylene glycol monoethyl ether alone was applied dropwise to a placebo group.

Two (2) days after the application, the number of living fleas, which were infesting the cats, was examined. When the examination was completed, all infested fleas were removed from the cats.

Both of 14 days and 28 days after the application, 50 test fleas (Ctenocephalides felis, adult ticks) were repeatedly deposited on a cat respectively. Two (2) days after the repeated deposition, the number of living ticks, which were infesting the dogs, was examined respectively. When each of the examination was completed, all infested ticks were removed from the dogs.

The infestation rate and control rate were calculated by the following formulae:

Method of calculating infestation rate and control rate at the initial stage (2 days) after the application:

Infestation rate (%) at X days after application=(number of living fleas at X days/number of living fleas before application)×100

Control rate (%) at X days after application {(infestation rate of test group before application infestation rate of test group at X days)/infestation rate of test group before application}×100

Method of calculating infestation rate and control rate at both 16 days and 30 days after the application:

Infestation rate (%) at X days after application=(number of living fleas at X days/number of living fleas repeatedly deposited)×100

Control rate (%) at X days after application {(infestation rate of placebo group at X days−infestation rate of test group at X days)/infestation rate of placebo group after X days}×100

In addition, if an infestation rate of a test group is higher than a placebo group, then the control rate is deemed to be 0%.

As a result, the present benzylamide compound (13) showed excellent tick control activities at a dose of 5, 10 and 20 mg/kg at 2 days, 16 days and 30 days after the application respectively (Table 4).

TABLE 4

| | | Tick control rates (%) at X days | | |
|---|---|---|---|---|
| | | after the application | | |
| | application rate | X = 2 | X = 16 | X = 30 |
| | (mg/kg) | (days) | (days) | (days) |
| Compound (13) | 5 | 92 | 97 | 97 |
| | 10 | 100 | 100 | 100 |
| | 20 | 100 | 100 | 100 |

### Test Example 8

### Pesticidal Activity on Ticks (Haemaphysalis longicornis) Dry Film Contact Application Test

Any one of the present benzylamide compounds (12), (13), (15), (28), (29), (30), (31), (32), (33), (34), (37) and (38) (16 μg) was dissolved in acetone (0.2 mL), and into a screw bottle (20 mL volume) this solution was dropwised and capped the bottle. Then the capped-bottle was made rolling so that the inner-surface of the screw bottle was uniformly coated with the solution. The acrew bottle was capped off and allowed to dry up for 2 hours. Into the screw bottle, test ticks (Haemaphysalis longicornis, non-blood-fed young ticks, 10 ticks/group) were added, and capped the bottle. Two (2) days later, the number of dead ticks was examined and the mortality was calculated by the following formula:

Mortality (%)=100×(number of dead ticks/number of ticks tested)

As a result, each of the present benzylamide compounds (12), (13), (15), (28), (29), (30), (31), (32), (33), (34), (37) and (38) showed a mortality of 90% or more.

### INDUSTRIAL APPLICABILITY

The controlling agent of the present invention has an excellent controlling effect on animal ectoparasites, and thus is useful for controlling animal ectoparasites.

1. An animal ectoparasite-controlling agent comprising a benzylamide compound represented by the formula (1): 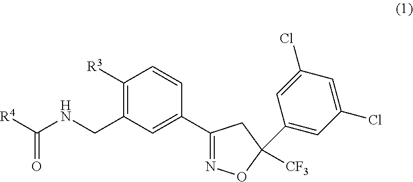
wherein R³ represents a hydrogen atom; a halogen atom; a cyano group; a C1-C6 alkyl group; a C1-C6 haloalkyl group; a C1-C6 alkoxy group or a C1-C6 haloalkoxy group; and R⁴ represents a C1-C6 alkyl group substituted with one or more C1-C6 alkoxy group(s); a C1-C6 alkyl group substituted with one or more cyano group(s); a C1-C6 alkyl group; a C1-C6 haloalkyl group or a C3-C6 cycloalkyl group,
as an active ingredient and an inert carrier.

2. The animal ectoparasite-controlling agent according to claim 1, which is in the form of an oral formulation or an external formulation for skin.

3. The animal ectoparasite-controlling agent according to claim 1, which is in the form of a liquid formulation.

4. The animal ectoparasite-controlling agent according to claim 1, which is in the form of a capsule formulation, a tablet or a chewable tablet.

5. The animal ectoparasite-controlling agent according to claim 1, wherein the animal ectoparasite is a flea or a tick.

6. A method for controlling an animal ectoparasite, which comprises applying an effective amount of
a benzylamide compound represented by the formula (1): 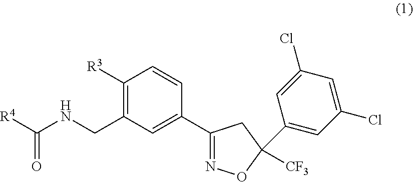
wherein R³ represents a hydrogen atom; a halogen atom; a cyano group; a C1-C6 alkyl group; a C1-C6 haloalkyl group; a C1-C6 alkoxy group or a C1-C6 haloalkoxy group; and R⁴ represents a C1-C6 alkyl group substituted with one or more C1-C6 alkoxy group(s); a C1-C6 alkyl group substituted with one or more cyano group(s); a C1-C6 alkyl group; a C1-C6 haloalkyl group or a C3-C6 cycloalkyl group,
to an animal.

7. The method for controlling an animal ectoparasite according to claim 6, wherein the present benzylamide compound is orally administered.

8. The method for controlling an animal ectoparasite according to claim 6, wherein the present benzylamide compound is externally applied to a skin.

9. The method for controlling an animal ectoparasite according to claim 8, wherein the present benzylamide compound is applied by spot-on application or pour-on application.

10. The method for controlling an animal ectoparasite according to claim 6, wherein the animal is a dog or a cat.

11. The method for controlling an animal ectoparasite according to claim 6, wherein the animal is a cow, a horse, a pig or a sheep.

12. The method for controlling an animal ectoparasite according to claim 6, wherein the animal ectoparasite is a flea or a tick.

13. The method for controlling an animal ectoparasite according to claim 7, wherein the animal is a dog or a cat.

14. The method for controlling an animal ectoparasite according to claim 8, wherein the animal is a dog or a cat.

15. The method for controlling an animal ectoparasite according to claim 9, wherein the animal is a dog or a cat.

16. The method for controlling an animal ectoparasite according to claim 7, wherein the animal is a cow, a horse, a pig or a sheep.

17. The method for controlling an animal ectoparasite according to claim 8, wherein the animal is a cow, a horse, a pig or a sheep.

18. The method for controlling an animal ectoparasite according to claim 9, wherein the animal is a cow, a horse, a pig or a sheep.

19. The method for controlling an animal ectoparasite according to claim 7, wherein the animal ectoparasite is a flea or a tick.

20. The method for controlling an animal ectoparasite according to claim 8, wherein the animal ectoparasite is a flea or a tick.
